(19) United States
(12) Patent Application Publication
(10) Pub. No.: US 2021/0213045 A1
(43) Pub. Date: Jul. 15, 2021

(54) NOVEL OPHTHALMIC COMPOSITION AND METHODS OF USE

(71) Applicant: Veloce BioPharma LLC, Fort Lauderdale, FL (US)

(72) Inventors: Joseph CAPRIOTTI DECIO, Arrecife, Las Palmas (ES); Kara CAPRIOTTI, Fort Washington, PA (US); Jesse PELLETIER, Miami, FL (US); Kevin STEWART, Christiansted, VI (US)

(73) Assignee: Veloce BioPharma LLC, Fort Lauderdale, FL (US)

(21) Appl. No.: 16/765,148

(22) PCT Filed: Mar. 13, 2018
(86) PCT No.: PCT/US2018/022124

§ 371 Date: May 18, 2020

Related U.S. Application Data
(63) Continuation of application No. 15/865,155, filed on Jan. 8, 2018.
Application No. 15/865,155 is a continuation of application No. 15/818,348, filed on Nov. 20, 2017, now abandoned.

(51) Int. Cl. A61K 31/79; A61K 47/38; A61P 31/04; A61P 31/12; A61P 31/10; A61K 9/00

(52) U.S. Cl. CPC A61K 31/79; A61K 47/38; A61K 9/0048; A61P 31/12; A61P 31/10; A61P 31/04

(57) ABSTRACT

Described are stable topical formulations useful in the treatment of viral infection, demodex infection, fungal infection and bacterial infection of the eye, and methods of using the compositions for treating viral infection, demodex infection, fungal infection and bacterial infection of the eye.

### BACKGROUND OF THE INVENTION

Millions of human patients suffer from viral infection or viral wart infection, demodex infection, fungal or yeast infection, or bacterial infection of the skin, including skin of the eye, eyelid or periocular region.

Infections of the eye or eyelid may occur via direct person-to-person skin contact, or indirectly through contaminated surfaces in a publicly accessed area, such as a public swimming pool or gymnasium. Exposure to the infectious agent can occur through minor abrasions and infection is promoted via maceration of the epithelia. Autoinoculation is common as well.

Verruca vulgaris, the medical term for wart, serves as an umbrella term for all warts. Warts can result from viral infections that are most often associated with Human Papilloma Virus (HPV) or Molluscum Contagiosum Virus (MCV) Non-genital varieties of skin warts occur in 20% of schoolchildren with equal frequency in both sexes.

Viral or viral wart infection, demodex infection, fungal or yeast infection, or bacterial infection of the eyelids, conjunctiva, cornea, ocular surface or Meibomian glands, can manifest as blepharitis and/or blepharoconjunctivitis or conjunctivitis—an infectious and inflammatory condition of the eyelid or ocular surface.

Blepharoconjunctivitis and blepharitis are commonly encountered conditions affecting approximately 15% of the population, and represent an inflammatory, infectious or mixed condition of the eyelids. Blepharitis may involve the dermis, eyelashes, tarsal conjunctiva, mucocutaneous junction or Meibomian glands and is most often caused by gram positive bacterial infection, such as Staphylococcus, Corynebacterium, and Propionibacterium species. However, other agents causing blepharitis include viral, demodex (mite), or yeast infections, seborrhea, rosacea, and hormonal dysregulation.

Left untreated, blepharitis may cause dry eye exacerbation, loss of cilia, corneal ulceration, and impart increased risk of endophthalmitis after cataract surgery. For facility in understanding, it is commonly compartmentalized into inflammation affecting the structures of the anterior, posterior lid margin or both.

Anterior blepharitis most commonly presents as anterior lid and lash crusting with or without the presence of collarettes. Other manifestations may also include skin or lash flaking associated with seborrhea or angular inflammation particular to Moraxella or virus.

Posterior blepharitis is also commonly referred to as Meibomian gland disease. Meibomian glands are responsible for the release of lipids into the tear film, effectively mitigating evaporative tear loss. Besides the chronic irritation, inflammation and erythema common to all blepharitis, the posterior variant may further be characterized by inspissation of the Meibomian glands, keratinization of orifices, telangiectasia, and posterior margin lid thickening. Bacterial lipases stemming from the ocular flora may also act upon Meibomian secretions creating free fatty acids which further disturb the ocular surface.

Current treatments for bacterial, demodex, fungal/yeast, and viral infections, including warts and ophthalmic conditions such as blepharitis, can be ineffective in that they treat only a subset of the causative agent of the infection. Many of the current treatments incorporate undesirable ingredients, such as steroids or other potentially harmful components.

A recent discovery by Capriotti, et al., has disclosed compositions comprising an iodophor such as povidone-iodine (PVP-I), as an active ingredient, and dimethyl sulfoxide (DMSO) were shown to be useful for treating fungal infections of the skin and nails. See, e.g., US Publication No. US2014/0205559 (Capriotti '559), which is incorporated herein by reference in its entirety.

Although a variety of organic solvents, including dimethyl sulfoxide (DMSO), are known to enhance the percutaneous absorption of certain medicaments, it has been long-accepted in the pharmaceutical arts that DMSO enhances penetration for small molecules or low molecular weight (LMW) compounds or drugs, only, and was not expected to enhance penetration of high molecular weight (HMW) compounds greater than about 10,000 Daltons, such as polymers, e.g., povidone-iodine. DMSO has only recently, and unexpectedly, been demonstrated to enhance penetration of povidone-iodine (PVP-I). PVP-I preparations range in molecular weights from 1,000 to 1,000,000 or more. Topical pharmaceutical compositions have been approved using only PVP grades K29-32. One acceptable PVP grade is PVP K30, which has a MW of 30,000 to 60,000 Daltons (average MW of about 40,000 Daltons). Accordingly, prior to the teachings of Capriotti '559, one skilled in the art would not employ DMSO in a topical pharmaceutical composition to enhance skin penetration of large molecules, polymers or high-molecular weight substances such as PVP-I.

Moreover, DMSO was understood and accepted in the art to be toxic to the eye and was not considered to be an acceptable ingredient in a composition intended for topical administration to the eye or periocular region. Therefore, not only was DMSO generally recognized as being unacceptable for use as a penetration enhancer for high molecular weight polymeric compounds, such as povidone-iodine, DMSO was particularly avoided as an ingredient for use in ophthalmic preparations, and especially avoided as an ingredient for topical ophthalmic preparations.

Further, although gel formulations comprising povidone-iodine and DMSO were mentioned, generally, in Capriotti '559 and related publications, it was discovered that certain formulations comprising povidone-iodine, DMSO, and a gelling agent were not stable for a sufficient period of time to provide a viable pharmaceutical product having an acceptable shelf-life. Only when particular amounts of povidone-iodine were combined with particular amounts of DMSO and particular amounts of gelling agent was stability observed to be sufficient to provide a viable pharmaceutically acceptable product having an acceptable and approvable shelf-life.

In US patent application, US Pub. No. 2017/0000819, Capriotti, et al., described topical ophthalmic gel compositions for treatment of certain ophthalmic conditions. However, it was shown that the compositions were not stable when concentrations of gelling agent, e.g., HEC, were less than 2% (w/w) of the composition. This result was consistent with the knowledge in the art that compositions comprising low concentrations of gelling agent were extremely difficult to stabilize. US Pub. No. 2017/0000819 described that stability and efficacy for topical gel compositions comprising PVP-I in DMSO was retained when gelling agent was provided at concentrations preferably between 2.5% and 5%.

A problem still exists in the art to provide topical ophthalmic preparations that contain lower concentrations of gelling agent but retain stability and efficacy for periods of time that translate into an acceptable shelf life for the composition. Providing topical ophthalmic compositions comprising concentrations of gelling agent less than 2% can be advantageous for proper administration or application of the product, residence time at the site of treatment, or aesthetic reasons (look/feel and patient acceptance of the product). Thus, discovery of a composition comprising PVP-I in DMSO, and water with or without an organic co-solvent, and a gelling agent concentration less than 2%, which is stable and effective for acceptable periods of time can represent a significant advancement to the medical arts, especially to the art of ophthalmic treatment.

Thus, the current invention is a significant advance in the art, and discloses the surprising and unexpected discovery that a topical ophthalmic gel composition comprising particular ingredients, namely, PVP-I, DMSO, and a gelling agent, in particular concentration combinations, can provide advantageous and unexpected results in the preparation and stabilization of ophthalmic formulations for the treatment of infection of the eye such as blepharitis, conjunctivitis, keratitis or other eye conditions.

### SUMMARY OF THE INVENTION

The present invention concerns a topical gel composition comprising an iodophor, a penetration enhancer, and a gelling agent, wherein the composition is particularly effective in treating viral, demodex, fungal/yeast, or bacterial infection that can cause warts or eye conditions, such as blepharitis. Thus, the subject invention further comprises a method of treating viral, demodex, fungal/yeast, or bacterial infection using a topical gel composition as disclosed herein. The composition can further comprise additional pharmaceutically acceptable excipients or solvents or co-solvents.

A composition of the subject invention preferably comprises active pharmaceutical ingredient (API) approved or allowed by the United States Food and Drug Administration (FDA) as acceptable for use in a pharmaceutical preparation for topical or ophthalmic administration. A preferred composition of the invention further comprises inactive ingredients or excipients that are FDA-approved for topical administration and/or ophthalmic administration. An FDA-approved API or inactive ingredient or excipient is referred to herein as “pharmaceutically acceptable.” Accordingly, a topical composition, formulation, or preparation of the subject invention comprising a pharmaceutically acceptable API, inactive ingredient or excipient is referred to herein as a “pharmaceutically acceptable” topical composition.

Similarly, an ophthalmic composition of the subject invention comprising FDA-approved active or inactive ingredients acceptable for use in an ophthalmic preparation, is referred to herein as a “pharmaceutically acceptable ophthalmic composition,” or “ophthalmically acceptable” composition, and comprises an API, an excipient, or a solvent which is “pharmaceutically acceptable” for ophthalmic use.

More particularly, the subject invention relates to a stable topical composition comprising an iodophor having a molecular weight of greater than 10,000 Daltons, e.g. povidone-iodine, such as povidone-iodine K30, dimethyl sulfoxide (DMSO), and a gelling agent, and optional additional pharmaceutically or ophthalmically acceptable excipients or solvents or co-solvents. In a preferred embodiment, the composition has a pH value of less than 4.5. Preferably, the pH value can be 4.4 or less, 4.3 or less, 4.2 or less, 4.1 or less, 4.0 or less, 3.9 or less, 3.8 or less, 3.7 or less, 3.6 or less, 3.5 or less, 3.4 or less, 3.3 or less, or 3.2 or less. In this embodiment, the pH value is not limited as long as it is less than 4.5. However, the lower limit of the pH value can be for example 0, 0.5, 1.0, 1.1, 1.2, 1.3, 1.4, 1.45, 1.5, 1.6, 1.7, 1.8, 1.9, 2.0, 2.1, 2.2, 2.3, 2.4, 2.5, 2.6, 2.7, 2.8, 2.9, 3.0, 3.1, or 3.2.

A composition of the subject invention can be useful in a method for treating viral infection or viral wart infection, demodex infection, fungal or yeast infection, or bacterial infection of the eye, eyelids, conjunctiva, cornea, ocular surface, Meibomian glands, or periocular region.

A topical gel composition of the subject invention is unexpectedly highly stable at room temperature in the presence of aqueous or anhydrous ingredients.

A topical ophthalmic gel composition of the subject invention can comprise about 0.1% to about 1.5% povidone-iodine (PVP-I); about 30% to about 99% dimethyl sulfoxide (DMSO); and about 0.5% to less than 2% gelling agent. The topical gel composition of the invention unexpectedly exhibits greater efficacy in treating skin infection or blepharitis, compared to a liquid composition substantially free of a gelling agent and comprising about 0.1% to about 1.5% povidone-iodine and about 30% to about 99% DMSO.

A preferred topical ophthalmic gel composition comprises about 0.15% to 1.5% povidone-iodine (PVP-I). A more preferred composition can comprise about 0.25% PVP-I to 0.5% PVP-I. A PVP-I grade of K30 is preferred for use in the subject composition.

A preferred topical ophthalmic gel composition comprises about 30% to about 99% DMSO. A more preferred composition can comprise about 30% to about 70% DMSO. A most preferred composition of the invention comprises about 40% to about 49% DMSO, and even more preferably, about 44% DMSO.

A preferred topical ophthalmic gel composition comprises about 0.25% to less than 2.0% gelling agent. A more preferred composition can comprise about 0.5% to about 1.75% gelling agent, about 0.75% to about 1.75% gelling agent, about 1.0% to about 1.5% gelling agent, and more preferably about 1.5% gelling agent.

A particularly useful topical ophthalmic gel composition which has been prepared for testing comprises 0.25% PVP-I; 44% DMSO; 0.25%-1.75% hydroxyethyl cellulose; and aqueous solvent, q.s. 100%. A preferred aqueous solvent is water or aqueous isotonic solution.

A gelling agent useful for preparing a topical ophthalmic gel composition of the invention can include, as is well known in the art, gum, agar, carrageenan, petrolatum, or a cellulosic polymer or the like. One preferred cellulosic polymer useful as a gelling agent is hydroxyethyl cellulose or salts thereof. An alternative cellulosic polymer gelling agent is hydroxymethyl cellulose or salts thereof. An alternative cellulosic polymer gelling agent is carboxymethyl cellulose or salts thereof.

A topical ophthalmic gel composition of the invention preferably comprises povidone-iodine, or PVP-I having an average molecular weight greater than 10,000. More preferably, the composition of the invention comprises PVP-I having an average molecular weight between about 20,000 to about 1,000,000. One preferred embodiment comprises PVP-I having an average molecular weight between about 30,000 to about 60,000, or greater. Each of the PVP-I ingredients having an average molecular weight ranging from 10,000 to about 1,000,000 is referred to herein, and means, “high molecular weight PVP-I,” or “HMW PVP-I.”

The topical ophthalmic gel composition is ophthalmically acceptable, comprising one or more ophthalmically acceptable ingredients. The ophthalmic gel composition embodiments described herein can advantageously can exhibit greater efficacy or stability in treating infectious conditions of the eye or eyelid, compared to a liquid composition substantially free of a gelling agent (or a gelling agent at concentrations below those required for forming a gel) and comprising about 0.1% to about 10% povidone-iodine and about 30% to about 99% DMSO.

A most preferred topical ophthalmic gel composition comprises a pharmaceutical grade povidone-iodine at a range of 0.15% to 0.5%; greater than 30% DMSO up to 90% DMSO; and a gelling agent at a range of 0.50% to less than 2.0%. All percentages are w/w unless otherwise specified. This most preferred composition can also include a co-solvent, such as polyethylene glycol (PEG). The preferred compositions of the subject invention are unexpectedly stable and efficacious for treating certain eye infections.

A preferred embodiment of the composition comprises an acidic solution, and more particularly, a gel solution, wherein the povidone-iodine is dissolved or solubilized in the final composition. Preferably, the subject composition is not an emulsion or suspension of particulates of ingredients in the gel or final composition.

Preferred embodiments of a topical gel composition of the invention are free of any additional API or anti-inflammatory drug, such as a steroid, e.g., corticosteroid, or non-steroidal anti-inflammatory drug (NSAID). Thus, a composition of the invention can be described as steroid-free, NSAID-free, steroid-free and NSAID-free, or anti-inflammatory-free (excepting any anti-inflammatory property exhibited by the specific ingredients comprising the subject composition.) A composition of the invention is advantageously useful for treatment of the described ophthalmic conditions without an additional anti-inflammatory, without a steroid, or without an NSAID present in the composition.

One preferred embodiment of a composition of the invention is a stable ophthalmically acceptable gel composition comprising

0.15% to 1.5% povidone-iodine (PVP-I);

30% to 97% dimethyl sulfoxide (DMSO);

0.75% to less than 2% hydroxyethyl cellulose; and

water or isotonic co-solvent;

wherein, the composition is formulated as a topical ophthalmic gel, free of additional anti-inflammatory drug, or being “anti-inflammatory-free”.

One preferred embodiment of a composition of the invention is a stable, ophthalmically acceptable gel composition comprising

0.15% to 1.5% povidone-iodine (PVP-I);

30% to 97% dimethyl sulfoxide (DMSO);

0.5% to less than 2.0% hydroxyethyl cellulose; and

water or isotonic co-solvent;

wherein, the composition is formulated as a topical ophthalmic gel, free of steroid (or being “steroid-free”.

One preferred embodiment of a composition of the invention is a stable, ophthalmically acceptable gel composition comprising

0.15% to 1.5% povidone-iodine (PVP-I);

30% to 97% dimethyl sulfoxide (DMSO);

0.25% to less than 2.0% hydroxyethyl cellulose; and

water or isotonic co-solvent;

wherein, the composition is formulated as a topical ophthalmic gel, free of steroid.

One preferred embodiment of a composition of the invention is a stable, ophthalmically acceptable gel composition comprising

0.15% to 1.5% povidone-iodine (PVP-I);

30% to 97% dimethyl sulfoxide (DMSO);

0.25% to 1.6% hydroxyethyl cellulose; and

water or isotonic co-solvent;

wherein, the composition is formulated as a topical ophthalmic gel, free of steroid.

One preferred embodiment of a composition of the invention is a stable, ophthalmically acceptable gel composition comprising

0.15% to 1.5% povidone-iodine (PVP-I);

30% to 97% dimethyl sulfoxide (DMSO);

0.25% to less than 2% methylcellulose; and

water or isotonic co-solvent;

wherein, the composition is formulated as a topical ophthalmic gel, free of corticosteroid.

One preferred embodiment of a composition of the invention is a stable, ophthalmically acceptable gel composition comprising

0.15% to 1.5% povidone-iodine (PVP-I);

30% to 97% dimethyl sulfoxide (DMSO);

1.0% to less than 2% hydroxyethyl cellulose; and

water or isotonic co-solvent;

wherein, the composition is formulated as a topical ophthalmic gel, free of NSAID.

A method according to the subject invention comprises, generally, one or more as-needed topical administrations or topical applications of a topical ophthalmic gel composition of the invention, namely, a topical composition comprising an iodophor, DMSO, and a gelling agent, to the site, until the ophthalmic infection is eliminated, or is substantially inhibited. In a preferred method, the subject gel composition is administered directly to the site of the infection as needed (PRN), preferably at least once per day (QD), or more preferably at least two times per day (BID) until results are seen, typically for about one week, up to about 24 weeks. Advantageously, the composition of the subject invention can be administered directly to the eye or periocular region and presents no toxicity to the eye.

A preferred embodiment of the invention comprises a method of treating an infectious condition of the eye or eyelid, comprising applying an effective amount of a stable, topical ophthalmic gel composition to a site of the infection to reduce or eliminate the infection.

The method of the invention can be useful in treating blepharitis, conjunctivitis, conical ulcer, HSV keratitis, conjunctival neoplasia, AC inflammation, post-operative endophthalmitis, and endophthalmitis after intravitreal or intracameral injection, which is caused by or associated with one or more infectious agents such as bacteria, demodex, fungus or yeast, or virus.

### DETAILED DESCRIPTION OF THE INVENTION

The present invention concerns a topical gel composition comprising an antiseptic agent, a penetration enhancer, and a gelling agent. Preferably, the composition comprises povidone-iodine as the antiseptic agent, dimethyl sulfoxide (DMSO) as the penetration enhancer, and a cellulosic gelling agent, such as hydroxyethyl cellulose (HEC), carboxymethyl cellulose, carboxymethyl cellulose sodium and other cellulosic polymers and salts thereof. The composition can, optionally, further comprise a lubricant or co-solvent, or other pharmaceutically or ophthalmically acceptable excipients. For example, a composition for treating an ophthalmic condition, such as blepharitis can include an ophthalmically acceptable excipient.

The subject composition is surprisingly useful for the treatment of viral, demodex, fungal/yeast or bacterial infection of the eye, eyelids, conjunctiva, cornea, ocular surface and Meibomian glands, which can cause blepharitis.

A specific but non-limiting example of a formulation of the invention providing a useful pharmaceutical preparation comprises solid PVP-I dissolved in DMSO with one or additional co-solvents in solution and prepared as a gel or semi-solid.

In another embodiment, DMSO can be added to aqueous solutions of PVP-I. In an example, DMSO can be present as a co-solvent with water in the range of 10%-99%. One embodiment of such a formulation can include a range of excipients such as water, or sodium chloride, sodium dihydrogen phosphate monohydrate, disodium hydrogen phosphate anhydrous and water, as well as other aqueous solutions or isotonic buffers known to those skilled in the art.

Percentages set forth herein are (w/w), with respect to the specified component in the overall composition, unless otherwise indicated. For example, a composition comprising 1% PVP-I and 45% DMSO has 1% PVP-I by weight, with respect to the total composition.

In an embodiment, a composition comprises povidone-iodine in the range of about 0.01% to about 15%. In another embodiment, a composition comprises povidone-iodine in the range between 0.05% and 12.5%. In another embodiment, a composition comprises povidone-iodine in the range between 0.05% and 10.0%. In another embodiment, a composition comprises povidone-iodine in the range between 0.1% and 10.0%. In another embodiment, a composition comprises povidone-iodine in the range between 0.1% and 1.0%. In another embodiment, a composition comprises povidone-iodine in the range between 0.15% and 1.5 5%. In another embodiment, a composition comprises povidone-iodine in the range between 0.25% and 0.5%.

In an embodiment, a composition comprises povidone-iodine of about 0.01%, about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.00%, about 1.1%, about 1.2%, about 1.3%, about 1.4%, about 1.5%, or any range determinable from the preceding percentages. All percentages are expresses as w/w unless otherwise specified.

In an embodiment, a composition comprises DMSO and PVP-I. In an embodiment, a composition consists essentially of DMSO and PVP-I and a gelling agent in an aqueous or anhydrous diluent. In an embodiment, a composition consists of DMSO and PVP-I and a gelling agent and a co-solvent in an aqueous or anhydrous diluent. In an embodiment, a composition is anhydrous. In an embodiment, a composition is substantially anhydrous. In an embodiment, a composition comprises a measurable amount of water.

In an embodiment, DMSO, e.g., anhydrous DMSO, is used in a composition. In an embodiment, substantially anhydrous DMSO is used in a composition. It will be understood by one of skill in the art that DMSO can be produced and/or obtained in differing grades, and that one of the variables among DMSO preparations of different grades is the water content. By way of example, DMSO may be completely anhydrous (also referred to herein simply as “anhydrous”), substantially anhydrous, or may contain water to a measurable degree. It will be understood that the amount of measurable water in a DMSO preparation may vary based on limitations of the instrumentation and techniques used to make such measurements.

In an embodiment, DMSO that is not completely anhydrous may be substantially anhydrous and contain water at a level below levels of detectability. In an embodiment, DMSO that is not completely anhydrous may contain water, wherein the water content is about at least 0.01%, about at least 0.02%, about at least 0.03%, about at least 0.04%, about at least 0.05%, about at least 0.06%, about at least 0.07%, about at least 0.08%, about at least 0.09%, about at least 0.1%, about at least 0.2%, about at least 0.3%, about at least 0.4%, about at least 0.5%, about at least 0.6%, about at least 0.7%, about at least 0.8%, about at least 0.9%, about at least 1.0%, about at least 1.5%, about at least 2.0%, about at least 2.5%, about at least 5%, about at least 7.5%, about at least 10%, about at least 12.5%, or greater. It will be understood that DMSO may contain one or more other impurities in addition to water.

In an embodiment, a composition comprises at least one of United States Pharmacopeia Convention (USP) grade DMSO, Active Pharmaceutical Ingredient (API) grade DMSO, analytical grade DMSO, and American Chemical Society (ACS) Spectrophotometric grade DMSO. In an embodiment, a composition comprises DMSO having <0.1% water by KF titration and >99.9% determined on an anhydrous basis.

As set forth above, the percent amount of DMSO in a composition is described in a weight-to-weight (w/w) ratio with respect to one or more other components of the composition, unless otherwise indicated. In an embodiment, the weight percent DMSO is the balance of the weight percent after addition of PVP-I. By way of a non-limiting example, a composition may comprise 1 weight percent (1%) PVP-I and 99 weight percent (99%) DMSO. It will be understood that in the foregoing example, the DMSO component of the composition may be completely anhydrous, substantially anhydrous, or may contain water to a measurable degree. In an embodiment, the weight percent DMSO is the balance of the weight percent after addition of PVP-I and any other components (e.g., co-solvent, water, additional active ingredient, etc.). In an embodiment, the weight percent DMSO is the balance of the weight percent after addition of iodophor and other components, if any. In an embodiment, the weight percent penetrant in a composition is the balance of the weight percent after addition of iodophor and other components, if any.

In an embodiment, a composition comprises DMSO in the range of 30% to 99.99%. In an embodiment, a composition comprises DMSO in the range of 35% to 99.99%. In another embodiment, a composition comprises DMSO in the range of 40% and 99.9%. In another embodiment, a composition comprises DMSO in the range of 30% and 97%. In another embodiment, a composition comprises DMSO in the range of 35% and 75%. In another embodiment, a composition comprises DMSO in the range of 40% and 50%. In another embodiment, a composition comprises DMSO in the range of 40% and 49%. In another embodiment, a composition comprises DMSO in the range of 43% and 45%. In another embodiment, a composition comprises DMSO at 44%.

In an embodiment, a composition comprises gelling agent in the range of less than 2%. In an embodiment, a composition comprises gelling agent in the range of 0.10% to less than 2.0%. In another embodiment, a composition comprises gelling agent in the range of 0.25% and 1.75%. In another embodiment, a composition comprises gelling agent in the range of 0.5% and 1.6%. In another embodiment, a composition comprises about 0.25% to about 1.95% gelling agent. In another embodiment, a composition comprises 1.75% gelling agent.

In an embodiment, a composition comprises a co-solvent in the range of 1% to 99.99%. In another embodiment, a composition comprises a co-solvent in the range of 5% and 99.9%. In another embodiment, a composition comprises a co-solvent in the range of 10% and 99.9%. In another embodiment, a composition comprises a co-solvent in the range of 20% and 99.9%. In another embodiment, a composition comprises a co-solvent in the range of 30% and 99.9%. In another embodiment, a composition comprises a co-solvent in the range of 40% and 99.9%. In another embodiment, a composition comprises a co-solvent in the range of 50% and 99.9%. In another embodiment, a composition comprises a co-solvent in the range of 60% and 99.9%. In another embodiment, a composition comprises a co-solvent in the range of 70% and 99.9%. In another embodiment, a composition comprises a co-solvent in the range of 80% and 99.9%, and in yet another embodiment, between 90% and 99.9%.

In an embodiment, a composition comprises a co-solvent at about 1%. In other embodiments, a composition comprises a co-solvent at about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

Examples of co-solvents include, but are not limited to, alcohols, silicones, polyethylene glycol, propylene glycol, glycerin, petrolatum, hydroxy methylcellulose, methylcellulose, and combinations thereof. In an embodiment, a co-solvent is polyethylene glycol.

In an embodiment, a composition comprises DMSO in the range of about 0.01% to 99.99% and further comprises at least one penetrant in the range of 0.01% to about 99.99%. In an embodiment, a composition comprises DMSO and further comprises at least one penetrant in the range of about 0.1% to about 50%. In another embodiment, a composition comprises DMSO and further comprises at least one penetrant in the range between about 5% and about 50%. In another embodiment, a composition comprises DMSO and further comprises at least one penetrant in the range between about 10% and about 99%. In an embodiment, a composition comprises DMSO, at least one co-solvent, and at least one penetrant. In an embodiment, a co-solvent is also a penetrant.

In an embodiment, where possible, compositions may include pharmaceutically acceptable salts of compounds in the composition. In an embodiment, compositions comprise acid addition salts of the present compounds. In an embodiment, compositions comprise base addition salts of the present compounds. As used herein, the term pharmaceutically acceptable salts or complexes refers to salts or complexes (e.g., solvates, polymorphs) that retain the desired biological activity of the parent compound and exhibit minimal, if any, undesired toxicological effects.

In various embodiments, the compositions encompassed herein comprise pharmaceutically acceptable excipients such as those listed in REMINGTON: THE SCIENCE AND PRACTICE OF PHARMACY 866-885 (Alfonso R. Gennaro ed. 19th ed. 1995; Ghosh, T. K.; et al. TRANSDERMAL AND TOPICAL DRUG DELIVERY SYSTEMS (1997), hereby incorporated herein by reference, including, but not limited to, protectives, adsorbents, demulcents, emollients, preservatives, antioxidants, moisturizers, buffering agents, solubilizing agents, skin-penetration agents, and surfactants.

Protectives and adsorbents include, but are not limited to, dusting powders, zinc stearate, collodion, dimethicone, silicones, zinc carbonate, aloe vera gel and other aloe products, vitamin E oil, allantoin, glycerin, petrolatum, and zinc oxide.

Demulcents include, but are not limited to, benzoin, hydroxypropyl cellulose, hydroxypropyl methylcellulose, and polyvinyl alcohol.

Emollients include, but are not limited to, animal and vegetable fats and oils, myristyl alcohol, alum, and aluminum acetate.

Preservatives include, but are not limited to, chlorine dioxide, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin.

Suitable antioxidants include, but are not limited to, ascorbic acid and its esters, sodium bisulfite, butylated hydroxytoluene, butylated hydroxyanisole, tocopherols, and chelating agents like EDTA and citric acid.

Suitable moisturizers include, but are not limited to, glycerin, sorbitol, polyethylene glycols, urea, and propylene glycol.

Suitable solubilizing agents include, but are not limited to, quaternary ammonium chlorides, cyclodextrins, benzyl benzoate, lecithin, and polysorbates.

Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methylpyrrolidone.

In an embodiment, a composition comprises PVP-I, DMSO, and polyethylene glycol and a gelling agent. In an embodiment, a composition comprises 0.25% PVP-I, 44% DMSO, 10% polyethylene glycol and 1.75% gelling agent. In an embodiment, a composition comprises PVP-I, DMSO, hydroxyethyl cellulose, propylene glycol and glycerin. In an embodiment, a composition comprises 2% PVP-I, about 40% DMSO, and 10-33% propylene glycol, a gelling agent and at least one additional inactive ingredient.

In one embodiment, the invention comprises DMSO 40-50% (w/w), 0.25%-0.55% PVP-I (w/w) and hydroxypropyl methylcellulose or hydroxymethyl cellulose or hydroxyethyl cellulose or carboxymethyl cellulose and salts thereof.

In one embodiment, the composition is a solution and can be formulated as a semi-solid, e.g., a gel, ointment or cream; tincture; foam; aerosol or another common pharmaceutical dosage form. In one embodiment, the composition is a 0.25% PVP-I144% DMSO solution dissolved in 1.75% gel, such as hydroxyethyl cellulose at pH less than 4.5.

### Stability

A. Visible Loss of Chromophore

Topical ophthalmic compositions comprising variations of the amounts and combinations of povidone-iodine, DMSO, and gelling agent ingredients were prepared to demonstrate the unpredictability of the stability for the subject compositions and to show the surprising result that lowering the pH of the gel compositions enabled the stabilization of lower concentrations of gelling agent than previously taught to be possible with similar PVP-I/DMSO compositions.

Compositions that are highly unstable and are thus not suitable or useful as a topical ophthalmic preparation, will lose color and appear colorless within about 72 hours of making the preparation. The colorless state is a result of loss of titratable iodine compared with the amount of iodine in the povidone-iodine starting material. No further confirmatory testing of titratable iodine is needed for compositions that are colorless within 72 hours of preparation.

Compositions were prepared with 0.15%, 0.25%, and 0.5% povidone-iodine. For these compositions, DMSO was provided in amounts of 0% or in an amount of 30% to 90%. Specific compositions comprising DMSO were prepared comprising 44% DMSO. Gelling agent was provided in amounts of 0%, 0.25%, 0.5%, 0.75%, 1.0%, 1.25%, 1.5%, 1.75% and 1.95%.

All of the prepared compositions comprising aqueous only PVP-I and water, with 0% DMSO and 0% gelling agent had lost substantially most of their color or were completely colorless within 72 hours at room temperature and were not determined to be stable for further use.

For preparations comprising 0.15% povidone-iodine and DMSO within the range of 30% to 90%, the compositions retained color and were stable when the gelling agent was provided at 0.75%. Long-term stability of the compositions which retained color for 72 hours or longer was confirmed by a modified USP assay for titratable iodine compared to povidone-iodine starting material. The stable compositions of the subject invention retained at least 85% of titratable iodine in the povidone-iodine starting material for up to 2 weeks when stored at room temperature and not shielded from light.

For preparations comprising 0.25% povidone-iodine and DMSO within the range of 30% to 90%, the compositions retained color and were stable when the amount of gelling agent was greater than or equal to 0.5%. Long-term stability of the compositions which retained color for 72 hours or longer was confirmed by modified USP assay for titratable iodine compared to povidone-iodine starting material. The stable compositions of the subject invention retained at least 85% of titratable iodine in the povidone-iodine starting material for up to 1 months when stored.

For preparations comprising 0.5% povidone-iodine and DMSO within the range of 30% to 90%, the compositions retained color and were stable when the amount of gelling agent was greater than or equal to 0.75%. Long-term stability of the compositions which retained color for 72 hours or longer was confirmed by modified USP assay for titratable iodine compared to povidone-iodine starting material. The stable compositions of the subject invention retained at least 85% of titratable iodine in the povidone-iodine starting material for up to 1 month at room temperature.

These experimental results demonstrate that stability of the specific combination of ingredients, and within specific range amounts, namely, 0.15% to 0.5% povidone-iodine; 30% to 90% DMSO, and 0.5% to 1.75% gelling agent, are stable whereas compositions which deviate from these specific ingredients [specifically those which do not contain gelling agent at least 0.5% (w/w)] in specific range amounts are unstable. A preferred gelling agent is a cellulosic polymer, such as hydroxyethyl cellulose (HEC), hydroxymethyl cellulose (HMC), hydroxypropyl methylcellulose (HPMC), and the like. A most preferred gelling agent is hydroxyethyl cellulose (HEC).

Tables showing results of stability experiments using selected compositions are provided below:

TABLE 1
Formulations with 0.25% PVP-I, 44%
DMSO and 0.50% HEC at pH 3.2HEC

| | HEC | % PVP-I Remaining |
|---|---|---|
| Sample | Concentration | of Initial After 2 |
| # | (w/w) | weeks at 25° C. |
| 1 | 0.75% | 91.35 |
| 2 | 0.75% | 85.09 |
| 3 | 0.75% | 87.38 |
| AVG | 0.75% | 87.94 |
| pH | 3.2 | 3.2 |

TABLE 2
Aqueous Formulations with 0.25% PVP-I,
44% DMSO and 1.0% HEC at pH 3.5

| | HEC | % PVP-I Remaining |
|---|---|---|
| Sample | Concentration | of Initial After 4 |
| # | (w/w) | weeks at 25 Deg C. |
| 1 | 1.0% | 92.0 |
| 2 | 1.0% | 89.27 |
| 3 | 1.0% | 89.13 |
| AVG | 1.0% | 90.0 |
| pH | 3.5 | 3.5 |

TABLE 3
Aqueous Formulations with 0.50% PVP-I,
44% DMSO and 0.5% HEC at pH 3.2

| | HEC | % PVP-I Remaining |
|---|---|---|
| Sample | Concentration | of Initial After 2 |
| # | (w/w) | weeks at 25 Deg C. |
| 1 | 0.50% | 91.00 |
| 2 | 0.50% | 92.15 |
| 3 | 0.50% | 93.44 |
| AVG | 0.50% | 92.23 |
| pH | 3.2 | 3.3 |

TABLE 4
Aqueous Formulations with 0.25% PVP-I,
44% DMSO and 1.0% HEC at pH 3.2

| | | % PVP-I Remaining |
|---|---|---|
| | HEC | of Initial After 18 |
| Sample | Concentration | months storage at |
| # | (w/w) | 3 Deg C.-8 Deg C. |
| 1 | 1.0% | 90.01 |
| 2 | 1.0% | 91.04 |
| 3 | 1.0% | 91.17 |
| AVG | 1.0% | 90.74 |
| pH | 3.2 | 3.4 |

TABLE 5
Aqueous Formulations with 0.50% PVP-I,
44% DMSO and 1.0% HEC at pH 3.5

| | | % PVP-I Remaining |
|---|---|---|
| | HEC | of Initial After 24 |
| Sample | Concentration | months storage at |
| # | (w/w) | 3 Deg C.-8 Deg C. |
| 1 | 1.0% | 88.90 |
| 2 | 1.0% | 89.03 |
| 3 | 1.0% | 91.04 |
| AVG | 1.0% | 89.65 |
| pH | 3.5 | 3.8 |

TABLE 6
Aqueous Formulations with 0.25% PVP-I,
44% DMSO and 0.75% HEC at pH 3.2

| | | % PVP-I Remaining |
|---|---|---|
| | HEC | of Initial After 24 |
| Sample | Concentration | months storage at |
| # | (w/w) | 3 Deg C.-8 Deg C. |
| 1 | 0.75% | 90.02 |
| 2 | 0.75% | 88.77 |
| 3 | 0.75% | 85.20 |
| AVG | 0.75% | 90.00 |
| pH | 3.5 | 3.9 |

TABLE 7
Formulations with 0.25% PVP-I,
44% DMSO and 1.0% HEC at pH 5.0

| | HEC | % PVP-I Remaining |
|---|---|---|
| Sample | Concentration | of Initial After 2 |
| # | (w/w) | weeks at 25 deg C. |
| 1 | 1.0% | 0 |
| 2 | 1.0% | 0 |
| 3 | 1.0% | 0 |
| AVG | 1.0% | 0 |
| pH | 5.0 | 5.2 |

TABLE 8
Formulations with 0.25% PVP-I, 44%
DMSO and 1.75% HEC at pH 6.0

| | HEC | % PVP-I Remaining |
|---|---|---|
| Sample | Concentration | of Initial After 2 |
| # | (w/w) | weeks at 25 Deg C. |
| 1 | 1.75% | 0 |
| 2 | 1.75% | 0 |
| 3 | 1.75% | 0 |
| AVG | 1.75% | 0 |
| pH | 6.0 | 6.3 |

TABLE 9
Formulations with 0.50% PVP-I,
44% DMSO and 0.5% HEC at pH 6.0

| | HEC | % PVP-I Remaining |
|---|---|---|
| Sample | Concentration | of Initial After 2 |
| # | (w/w) | weeks at 25 Deg C. |
| 1 | 0.50% | 0 |
| 2 | 0.50% | 0 |
| 3 | 0.50% | 0 |
| AVG | 0.50% | 0 |
| pH | 6.0 | 6.2 |

TABLE 10
Formulations with 0.25% PVP-I, 44%
DMSO and 1.50% HEC at pH 6.0

| | HEC | % PVP-I Remaining |
|---|---|---|
| Sample | Concentration | of Initial After 72 |
| # | (w/w) | hours at 25 Deg C. |
| 1 | 1.50% | 0 |
| 2 | 1.50% | 0 |
| 3 | 1.50% | 0 |
| AVG | 1.50% | 0 |
| pH | 6.0 | 6.2 |

TABLE 11
Formulations with 0.50% PVP-I,
44% DMSO and 0.5% HEC at pH 6.0

| | HEC | % PVP-I Remaining |
|---|---|---|
| Sample | Concentration | of Initial After 2 |
| # | (w/w) | weeks at 25 Deg C. |
| 1 | 0.50% | 0 |
| 2 | 0.50% | 0 |
| 3 | 0.50% | 0 |
| AVG | 0.50% | 0 |
| pH | 6.0 | 6.2 |

TABLE 12
Formulations with 0.50% PVP-I, 44%
DMSO and 1.75% HEC at pH 6.0

| | HEC | % PVP-I Remaining |
|---|---|---|
| Sample | Concentration | of Initial After 16 |
| # | (w/w) | weeks at 25 Deg C. |
| 1 | 1.75% | 0 |
| 2 | 1.75% | 0 |
| 3 | 1.75% | 0 |
| AVG | 1.75% | 0 |
| pH | 6.0 | 6.4 |

TABLE 13
Formulations with 0.25% PVP-I, 44%
DMSO and 1.75% HEC at pH 6.0

| | HEC | % PVP-I Remaining |
|---|---|---|
| Sample | Concentration | of Initial After 2 |
| # | (w/w) | weeks at 25 Deg C. |
| 1 | 1.75% | 0 |
| 2 | 1.75% | 0 |
| 3 | 1.75% | 0 |
| AVG | 1.75% | 0 |
| pH | 6.0 | 6.1 |

TABLE 14
Formulations with 0.25% PVP-I,
44% DMSO and 1.5% HEC at pH 4.4

| | HEC | % PVP-I Remaining |
|---|---|---|
| Sample | Concentration | of Initial After 4 |
| # | (w/w) | weeks at 25 Deg C. |
| 1 | 1.5% | 90% |
| 2 | 1.5% | 92% |
| 3 | 1.5% | 95% |
| AVG | 1.5% | 92% |
| pH | 4.4 | 4.4 |

TABLE 15
Formulations with 0.25% PVP-I, 44%
DMSO and 1.5% HEC at pH 1.45

| | HEC | % PVP-I Remaining |
|---|---|---|
| Sample | Concentration | of Initial After 4 |
| # | (w/w) | weeks at 25 Deg C. |
| 1 | 1.5% | 95% |
| 2 | 1.5% | 97% |
| 3 | 1.5% | 96% |
| AVG | 1.5% | 96% |
| pH | 1.45 | 1.50 |

TABLE 16
Formulations with 0.25% PVP-I, 44%
DMSO and 1.5% HEC at pH 1.45

| | HEC | % PVP-I Remaining |
|---|---|---|
| Sample | Concentration | of Initial After 2 |
| # | (w/w) | weeks at 35 Deg C. |
| 1 | 1.5% | 90% |
| 2 | 1.5% | 90% |
| 3 | 1.5% | 90% |
| AVG | 1.5% | 90% |
| pH | 1.60 | 1.50 |

In one embodiment, the formulations are stable at room temperature 25° C. for at least 6 months, 12 months, 18 months and 24 months. Stability is defined as where the final PVP-I concentration is at least 85%-120%, according to the USP titration method for povidone-iodine, of the labeled concentration.

In one embodiment, the formulations are stable at temperature 2-8° C. for at least 6 months, 12 months, 18 months and 24 months. Stability is defined as where the final PVP-I concentration is at least 85%-120%, according to the USP method of the labeled concentration (e.g. if the label is 2% PVP-I providing for 0.2% iodine, therefore 90% would be 0.18% elemental iodine).

In one embodiment, the formulations are stable at room temperature −10 to −25° C. for at least 6 months, 12 months, 18 months and 24 months. Stability is defined as where the final PVP-I concentration is at least 85%-120%, according to the USP method of the labeled concentration (e.g. if the label is 2% PVP-I providing for 0.2% iodine, therefore 90% would be 0.18% elemental iodine).

In one embodiment, the formulations are stable at room temperature 15-30° C. for at least 6 months, 12 months, 18 months and 24 months. Stability is defined as where the final PVP-I concentration is at least 85%-120%, according to the USP method, of the labeled concentration (e.g. if the label is 2% PVP-I providing for 0.2% iodine, therefore 90% would be 0.18% elemental iodine).

In one embodiment, the formulations are stable at room temperature 40° C. for at least 1 months, 3 months, 6 months, 12 months, 18 months and 24 months. Stability is defined as where the final PVP-I concentration is at least 85%-120%, according to the USP method of the labeled concentration (e.g. if the label is 2% PVP-I providing for 0.2% iodine, therefore 90% would be 0.18% elemental iodine).

### Methods of Preparation and Use

It is known to one of skill in the art that PVP-I aqueous solutions are difficult to stabilize at low PVP-I concentrations over a long period of time. By way of a non-limiting example, at concentrations of PVP-I less than about 0.6% (w/w, aqueous), PVP-I aqueous solutions rapidly decay to yield complex mixtures of iodinated and iodine-free constituents.

As described herein, it was surprisingly found that in the aprotic DMSO solvent system encompassed by the disclosure set forth herein, PVP-I gel solutions as low as 0.15% can be easily prepared and maintained as stable compositions for long periods of time. Also, as previously described, hydrated DMSO solutions prepared from aqueous PVP-I and sufficient (about 2% or greater) gelling agent, demonstrate increased stability for the PVP-I component over at least 12 months at room temperature. It is surprisingly demonstrated in this application, for the first time, and against the teaching of the prior art, that acidic solutions of gelling agent, specifically HEC, can be employed to stabilize low concentration PVP-I/DMSO gels even when as little as 0.5% gelling agent HEC is employed.

In an embodiment, a composition comprises dry, solid or powdered PVP-I dissolved or suspended in a composition comprising or consisting of DMSO. In another embodiment, DMSO is added to an aqueous preparation comprising or consisting of PVP-I.

Based on the disclosure herein, one of ordinary skill in the art will understand how to prepare a composition to arrive at the desired amounts of iodine, iodophor, and DMSO, among other possible components of the composition encompassed herein, without undue experimentation.

By way of a non-limiting example, a therapeutically-effective pharmaceutical composition is prepared using solid PVP-I, which is dissolved or suspended in DMSO. In an aspect, the composition is anhydrous. In an aspect, the composition is substantially anhydrous. In another embodiment, DMSO can be added to aqueous solutions of PVP-I to prepare a therapeutically-effective pharmaceutical composition. In an embodiment, DMSO is used in the range of 30%-99% as a co-solvent with water and other non-aqueous co-solvents. In an embodiment, a formulation includes one or more excipients. By way of a non-limiting example, excipients include, but are not limited to, sodium chloride, sodium dihydrogen phosphate monohydrate, disodium hydrogen phosphate anhydrous and water, as well as others known to those skilled in the art.

In an embodiment, a composition is prepared by adding PVP-I (w/v, aqueous) to pure DMSO q.s. to yield a resulting solution of 0.15-1.5% PVP-I (w/w) with DMSO. In another embodiment, compositions are prepared by dissolving solid PVP-I in pure DMSO q.s to obtain any of 0.1%, 0.2%, 0.25%, 0.3%, 0.35%, 0.4%, 0.45%, or 0.5% PVP-I (w/w) compositions, with DMSO as the solvent. In yet another embodiment, compositions are prepared by dissolving solid PVP-I in pure DMSO q.s to obtain any composition set forth, described, and/or encompassed herein. Similar compositions comprising aqueous PVP-I (with and without excipients commonly used and/or known in the art) and DMSO can be prepared from a stock 10% PVP-I aqueous solution and pure DMSO. Gelling agent can be added at an amount up to less than 2.0% (w/w) of the final concentration of the composition, preferably between about 0.5% and 1.75%, between about 0.5% and 1.0%, and 1.5%. Acidification of the compositions can be achieved by adding any number of commonly employed acidifying agents including but not limited to HCl,

It will be understood by the skilled artisan, however, that any starting composition of PVP-I, solid or liquid, may be used when the appropriate dilutions and adjustments are made to result in the desired final PVP-I concentration. Similarly, any starting composition of iodophor or elemental iodine may be used when the appropriate dilutions and adjustments are made to result in the desired final iodophor or elemental iodine concentration, respectively.

It will be understood, based on the disclosure set forth herein, in view of the skill in the art, that specific dosage for compounds and compositions encompassed herein may be determined empirically through clinical and/or pharmacokinetic experimentation, and that such dosages may be adjusted according to pre-specified effectiveness and/or toxicity criteria. It will also be understood that a specific dosage and treatment regimen for any particular patient will depend upon a variety of factors, including the activity of the specific compounds employed, the characteristics of the patient, drug combination, the judgment of the treating physician and the nature and severity of the particular disease or condition being treated.

In an embodiment, a therapeutic composition is prepared by optimizing one or more compounds for use in a dosage form different than that which is typically used for the compound. In an embodiment, a compound that is not typically administered in a topical dosage form is developed for use in a topical dosage form. The chemical and biological assays required for such development are known to one of skill in the art. The disclosure herein provides the skilled artisan with the guidance as to how to prepare such compounds and compositions comprising such compounds.

In an embodiment, a method of treating a subject having an ocular surface disease complicated by microbial colonization and/or infection includes administration of a composition set forth, described, and/or encompassed herein to treat the ocular surface disease, and the treatment of the ocular surface disease includes at least one of preventing or slowing the progression of the infection, preventing the spread of the infection, eradicating at least some of the infection, and eradicating the entire infection.

In an embodiment, a therapeutic composition is administered on a schedule once a day. In an embodiment, a therapeutic composition is administered twice a day. Typically, a gel composition of the subject invention can be administered as a ribbon having a length of about 1 cm to about 5 cm, up to 1 cm in diameter, onto the eye, under the eyelid, or at the periocular region of the eye to be treated.

In an embodiment, a therapeutic composition is administered three times a day, four times a day, five times a day, or more. In an embodiment, a therapeutic composition is administered less frequently than once a day. In an embodiment, a therapeutic composition is administered once every two days, once every three days, once every four days, once every five days, once every six days, or once every seven days. In an embodiment, a therapeutic composition is administered less frequently than once a week. In an embodiment, a therapeutic composition is administered once a month. In an embodiment, a therapeutic composition is administered twice a month.

In an embodiment, a therapeutic dosing regimen is continued for at least one day, at least two days, at least three days, at least four days, at least five days, at least six days, or at least seven days. In an embodiment, a therapeutic dosing regimen is continued for at least one week, at least two weeks, at least three weeks, at least four weeks, at least six weeks, at least eight weeks, at least ten weeks, at least twelve weeks, at least fourteen weeks, or at least sixteen weeks. In an embodiment, a therapeutic dosing regimen is continued for at least one month, at least two months, at least three months, at least four months, at least five months, at least six months, at least nine months, or at least twelve months.

The invention is further described by the following examples. In an aspect, the following examples demonstrate effective and/or successful treatment of the identified conditions using compositions and methods encompassed by the present disclosure. It should be recognized that variations based on the inventive features are within the skill of the ordinary artisan, and that the scope of the invention should not be limited by the examples. To properly determine the scope of the invention, an interested party should consider the claims herein, and any equivalent thereof. The entire disclosure of international patent applications, PCT/US2012/036942 and PCT/US2012/065298 are hereby incorporated herein by reference as if fully set forth herein. In addition, all citations herein are incorporated by reference, and unless otherwise expressly stated, all percentages are by weight/weight.

Additional examples of useful compositions described for this invention include the formulation of creams, petrolatum balms, salves, sprays, and other formulations well known to those in the art suitable for topical administration to the ocular surface or are “ophthalmically acceptable” compositions.

### EXAMPLES

The Examples and description provided herein are not intended as, and are not, limiting to the breadth of protection afforded within the full scope and spirit of the invention as described.

The results of clinical treatments using a composition according to the subject invention are shown in Table 14, below:

TABLE 14
Clinical Results in Patients Treated with 0.25% PVP-I, 44.0% DMSO, 1.0% HEC and water (pH = 3.5)

| | | Blepharitis Symptoms | | Blepharitis Signs | | Conjunctival Inflammation | |
|---|---|---|---|---|---|---|---|
| Age | Sex | Pre Tx | 1 M Post Tx | Pre Tx | 1 M Post Tx | Pre Tx | 1 M Post Tx |
| 27 | M | Itching/Burning | Resolved | Posterior | Improved meibum | None | None |
| | | | | Blepharitis | viscosity | | |
| 88 | M | Foreign sensation | Resolved | Mixed | Improved lid erythema, | 2+ | None |
| | | | | Blepharitis | scurf, meibum viscosity; | | |
| | | | | | resolved lid edema | | |
| 83 | F | Tearing/Burning | Resolved | Mixed | Improved lid erythema, | 1+ | None |
| | | | | Blepharitis | scurf, meibum viscosity; | | |
| | | | | | resolved lid edema | | |
| 67 | F | Burning | Improved | Mixed | Improved eyelid | 1+ | None |
| | | | | Blepharitis | erythema; resolved lid | | |
| | | | | | edema | | |
| 88 | M | Discharge/Itching | Resolved | Mixed | Improved lid erythema, | 2+ | None |
| | | | | Blepharitis | scurf, meibum viscosity; | | |
| | | | | | resolved lid edema | | |
| 78 | F | Blurry vision | Improved | Mixed | Improved scurf, meibum | 1+ | None |
| | | | | Blepharitis | viscosity; resolved lid | | |
| | | | | | edema and lid erythema | | |
| 76 | M | Blurry | Resolved | Mixed | Resolved lid edema, lid | 1+ | None |
| | | vision/Tearing | | Blepharitis | erythema; scurf; | | |
| | | | | | improved meibum | | |
| | | | | | viscosity | | |
| 79 | M | Stinging/Burning | Resolved | Mixed | Resolved lid edema, lid | 1+ | None |
| | | | | Blepharitis | erythema; scurf; | | |
| | | | | | improved meibum | | |
| | | | | | viscosity | | |
| 92 | M | Tearing/Burning | Improved | Mixed | Improved lid erythema, | 1+ | 1+ |
| | | | | Blepharitis | scurf, meibum viscosity; | | |
| | | | | | resolved lid edema | | |
| 56 | F | Burning/Tearing | Resolved | Anterior | Resolved lid edema, lid | None | None |
| | | | | Blepharitis | erythema; scurf | | |
| 69 | F | Tearing/Redness | Resolved | Posterior | Improved meibum | 1+ | None |
| | | | | Blepharitis | viscosity | | |
| 73 | M | Blurry vision | Resolved | Posterior | Improved meibum | None | None |
| | | | | Blepharitis | viscosity | | |
| 82 | F | Burning/Tearing | Resolved | Anterior | Resolved lid collarettes, | None | None |
| | | | | Blepharitis | edema, erythema | | |
| 31 | F | Burning | Resolved | Posterior | Improved meibum | None | None |
| | | | | Blepharitis | viscosity | | |
| 73 | F | Burning/Itching | Resolved | Mixed | Improved lid erythema, | 1+ | None |
| | | | | Blepharitis | scurf, meibum viscosity; | | |
| | | | | | resolved lid edema | | |
| 73 | F | Tearing/Burning | Resolved | Mixed | Improved lid erythema, | 2+ | None |
| | | | | Blepharitis | scurf; resolved lid edema | | |
| 72 | F | Stinging/Burning | Improved | Mixed | Improved lid erythema, | 1+ | 1+ |
| | | | | Blepharitis | scurf, edema | | |

### TREATMENT EXAMPLES

Below are examples of clinical treatments that can be carried out using a composition of the subject invention, illustrating the positive clinical responses in young, middle-aged, and elderly patients, both male and female, that can result from such treatments, which would have been unexpected in view of the prior art, but predicted based on the stability and efficacy demonstrated by the compositions developed and tested in accordance with this disclosure.

(1) An 82-year-old female may present with visible, bilateral eye discomfort, pain, redness and discharge. Ophthalmoscopy would reveal bilateral 2+ erythematous conjunctiva with mild chemosis and discharge. Eyelid eversion would demonstrate a mixed papillary and follicular reaction. The patient would receive a diagnosis of bacterial conjunctivitis and a dosing regimen would be employed whereby an approximately ½-inch ribbon or gel drop of a composition comprising 0.25% PVP-I and 44% DMSO with 1.5% HEC is administered directly to the surface of the infected eye between corneal surface and the lower eyelid. The composition would be administered two times per day for a period of seven days. After three days of treatment, both the chemosis and erythema would begin to improve. By the fifth day, the residual inflammation would abate, and the ocular examination would be unremarkable. The patient would endorse complete symptomatic relief and return to her normal function.

(2) An 87-year-old female would present with recurrent visible, bilateral eye discomfort, pain, redness and greenish discharge. She has been previously admitted to the ICU where she was diagnosed with a multi-drug resistant MRSA ocular infection. Ophthalmoscopy would reveal bilateral 4+ erythematous conjunctiva with mild chemosis and discharge. Eyelid inspection would be positive for abundant mixed blepharitis with eyelid edema and erythema. The patient would receive a diagnosis of MRSA, multi-drug resistant bacterial blepharoconjunctivitis and a dosing regimen would be employed whereby an approximately ½-inch ribbon or gel drop of a composition comprising 0.50% PVP-I and 44% DMSO with 1.0% HEC is administered directly to the surface of the infected eye between corneal surface and the lower eyelid. The composition would be administered two times per day for a period of ten days. After three days of treatment, both the chemosis and erythema would begin to improve. By the sixth day, the eyelid edema and erythema would begin to abate. On the eighth day, the residual inflammation resolves, and the ocular examination would be unremarkable. The patient endorses complete symptomatic relief, returns to her normal functions and does not suffer a recurrence.

(3) A 77-year-old male would present with visible, bilateral eye discomfort, itching, redness and eyelid inflammation. Ophthalmoscopy would reveal bilateral trace erythematous conjunctiva without chemosis or discharge. Eyelid inspection would reveal 3+ eyelid edema with erythema and non-cylindrical collarettes. There would be 2+ meibomian gland inspissation and plugging. The patient would receive a diagnosis of mixed anterior and posterior blepharitis and a dosing regimen would be employed whereby an approximately ½-inch ribbon or gel drop of a composition comprising 0.25% PVP-I and 44% DMSO with 0.75% HEC would be administered directly to the skin of the infected eyelid at the lower eyelid margin. The composition would be administered two times per day for a period of fourteen days. After five days of treatment, both the chemosis and erythema would begin to improve. By the tenth day, the posterior eyelid meibum has decreased in viscosity and plugging has resolved. At the end of the two-week treatment cycle, the residual inflammation abates, and the ocular examination would be unremarkable. The patient endorses complete symptomatic relief and returns to his normal functions.

(4) A 72-year-old female would present with visible, bilateral eye discomfort, itching, redness and eyelid inflammation. She has been unsuccessfully treated for blepharitis in the past. Ophthalmoscopy would reveal bilateral trace erythematous conjunctiva without chemosis or discharge. Eyelid inspection would reveal 3+ eyelid edema with erythema and cylindrical collarettes at the base of the eyelashes. Wet mount prep under 25× magnification would reveal the presence of Demodex folliculorum. The patient would receive a diagnosis of Demodex blepharitis and a dosing regimen would be employed whereby an approximately ½-inch ribbon or gel drop of a composition comprising 0.50% PVP-I and 44% DMSO with 1.75% HEC would be administered directly to the skin of the infected eyelid at the lower eyelid margin. The composition would be administered two times per day per for a period of fourteen days. After five days of treatment, both the chemosis and erythema would begin to improve. By the seventh day, the cylindrical collarettes are no longer present. At the end of the two-week treatment cycle, the residual inflammation abates, and the ocular examination would be unremarkable. The patient endorses complete symptomatic relief and returns to her normal functions.

(5) A 68-year-old male would present with visible, bilateral eye discomfort, itching, crusting, redness and eyelid inflammation. He would present also with rhinophyma and intermittent facial flushing. Ophthalmoscopy would reveal bilateral trace erythematous conjunctiva without chemosis or discharge. Eyelid inspection would reveal 2+ eyelid edema with erythema and scurf. 2+ meibomian gland plugging would be also present along with tortuous posterior eyelid margin telangiectasias. The patient would receive a diagnosis of rosacea blepharitis and a dosing regimen would be employed whereby an approximately ½-inch ribbon or gel drop of a composition comprising 0.25% PVP-I and 44% DMSO with 1.25% HEC would be administered directly to the skin of the infected eyelid. The composition would be administered two times per day for a period of fourteen days. After five days of treatment, both the chemosis and erythema would begin to improve. By the tenth day, the posterior eyelid meibum has decreased in viscosity and plugging has resolved. At the end of the two-week treatment cycle, the residual inflammation abates and the lid margin telangiectasias have attenuated significantly. The ocular examination would be otherwise unremarkable. The patient endorses symptomatic relief and returns to his normal functions.

(6) A 52-year-old male would present with visible, bilateral eye discomfort, pain, redness and clear discharge. He endorses recent contact with others in his family who have contacted pink eye. Ophthalmoscopy would reveal bilateral erythematous conjunctiva with mild chemosis and discharge. There are no corneal subepithelial infiltrates or pseudomembranes. Lid eversion would reveal an abundant follicular reaction and a pre-auricular node is palpable. The patient would receive a diagnosis of viral conjunctivitis and a dosing regimen would be employed whereby an approximately ½-inch ribbon or gel drop of a composition comprising 0.25% PVP-I and 44% DMSO with 1.75% HEC would be administered directly to the surface of the infected eye between corneal surface and the lower eyelid. The composition would be administered two times per day per for a period of ten days. After three days of treatment, both the chemosis and erythema would begin to improve. By the fifth day, the residual inflammation abates, and the ocular examination would be unremarkable. The patient continues the medicine for the full period of seven days in order to decrease the chances of viral shedding. He endorses complete symptomatic relief and returns to normal function.

(7) A 27-year-old female would present with visible, bilateral eye discomfort, pain, itching, redness and clear discharge. She endorses recent contact with others in her family who have contacted pink eye. Ophthalmoscopy would reveal bilateral erythematous conjunctiva with mild chemosis and discharge. Corneal subepithelial infiltrates are present and an early pseudomembrane would be visualized in the lower palpebral fornix. Lid eversion would reveal an abundant follicular reaction and a pre-auricular node would be palpable. The patient receives a diagnosis of epidemic keratoconjunctivitis secondary to adenovirus infection and a dosing regimen would be employed whereby an approximately ½-inch ribbon or gel drop of a composition comprising 0.25% PVP-I and 44% DMSO with 1.25% HEC would be administered directly to the surface of the infected eye between corneal surface and the lower eyelid. The composition would be administered two times per day for a period of ten days. After three days of treatment, both the chemosis and erythema would begin to improve. By the fifth day, the residual inflammation abates, and the ocular examination would be unremarkable. The patient continues the medicine for the full period of seven days in order to decrease the chances of viral shedding. She endorses complete symptomatic relief and returns to normal function.

(8) A 35-year-old male would present with visible, unilateral eye discomfort, redness and discharge after being struck in the eye with vegetative matter. Ophthalmoscopy would reveal 2+ erythematous conjunctiva with mild chemosis and discharge. Corneal inspection would be positive for a staining 2×2 mm corneal ulceration with infiltrate demonstrating crenate, feathered edges. Corneal culture would reveal the growth of filamentous fungus. The patient receives a diagnosis of trauma related fungal corneal ulcer and a dosing regimen is employed whereby an approximately ½-inch ribbon or gel drop of a composition comprising 0.50% PVP-I and 44% DMSO with 1.75% HEC would be administered directly to the surface of the infected eye between corneal surface and the lower eyelid. The composition would be administered four times per day for a period of one month. After five days of treatment, both the chemosis and erythema would begin to improve. By second week, the corneal infiltrate would begin to diminish, the cornea has re-epithelialized and there are no limbal white cells present. At the end of three weeks, a small, pinpoint infiltrate still remains and by the end of the month the cornea would be clear and compact centrally and an inactive scar remains. All residual inflammation abates, and the ocular examination would be unremarkable. The patient endorses complete symptomatic relief and returns to his normal functions.

(9) A 22-year-old female would present with visible, unilateral eye discomfort, redness and discharge after sleeping overnight in her contact lenses. Ophthalmoscopy would reveal 2+ erythematous conjunctiva with mild chemosis and discharge. Corneal inspection would be positive for a staining 3×2 mm corneal ulceration with infiltrate, suppuration and 25% stromal thinning Corneal culture would reveal the growth of Pseudomonas spp. The patient receives a diagnosis of a bacterial corneal ulcer and a dosing regimen would be employed whereby an approximately ½-inch ribbon or gel drop of a composition comprising 0.50% PVP-I and 44% DMSO with 0.75% HEC would be administered directly to the surface of the infected eye between corneal surface and the lower eyelid. The composition would be administered four times per day for a period of three weeks. After five days of treatment, both the chemosis and erythema would begin to improve. By second week, the corneal infiltrate would begin to diminish, the cornea has re-epithelialized and there are no limbal white cells present. At the end of three weeks, the cornea would be clear and compact centrally with an inactive cicatrix in the area of the previous ulcer. The ocular examination would be otherwise unremarkable. The patient endorses complete symptomatic relief and returns to her normal functions.

(10) A 27-year-old female would present with visible, unilateral eye discomfort, redness and discharge after sleeping overnight in her contact lenses and using dubious contact lens solution. Ophthalmoscopy would reveal 2+ erythematous conjunctiva with mild chemosis and discharge. Corneal inspection would be positive for a ring ulceration with pain out of proportion to examination. Corneal culture would reveal the growth of Acanthamoeba spp. The patient receives a diagnosis of an Acanthamoeba corneal ulcer and a dosing regimen would be employed whereby an approximately ½-inch ribbon or gel drop of a composition comprising 0.50% PVP-I and 44% DMSO with 1.50% HEC would be administered directly to the surface of the infected eye between corneal surface and the lower eyelid. The composition would be administered four times per day for a period of three weeks. After five days of treatment, both the chemosis and erythema would begin to improve. By second week, the corneal ring infiltrate would begin to diminish, the cornea has re-epithelialized and there are no peripheral limbal white cells present. At the end of three weeks, the cornea would be clear and compact centrally. The ocular examination would be otherwise unremarkable. The patient endorses complete symptomatic relief and returns to her normal functions.

(11) A 45-year-old male business professional would present with visible, unilateral eye discomfort, pain and redness. He remembers a similar episode occurring a few years ago. Ophthalmoscopy would reveal 2+ erythematous conjunctiva. Corneal inspection would be positive for a staining dendritic corneal ulceration. The patient receives a diagnosis of herpes simplex virus epithelial keratitis and a dosing regimen would be employed whereby an approximately ½-inch ribbon or gel drop of a composition comprising 0.50% PVP-I and 44% DMSO with 1.25% HEC would be administered directly to the surface of the infected eye between corneal surface and the lower eyelid. The composition would be administered four times per day for a period of two weeks. After five days of treatment, ocular erythema would begin to improve and the dendrites attenuate. By the second week dendrites have completely resolved, the cornea has re-epithelialized and would be clear and compact. The ocular examination would be otherwise unremarkable. The patient endorses complete symptomatic relief and returns to his normal functions.

(12) An 81-year-old female would present with visible, unilateral eye discomfort, pain, redness and decreased vision. On visual inspection, an excoriated dermatomal rash involving both the face and scalp would be present. Ophthalmoscopy would reveal 2+ erythematous conjunctiva without discharge. Corneal inspection would be positive for raised epithelial dendritiform lesions. The patient receives a diagnosis of herpes zoster ophthalmicus and a dosing regimen would be employed whereby an approximately ½-inch ribbon or gel drop of a composition comprising 0.50% PVP-I and 44% DMSO with 1.00% HEC would be administered directly to the surface of the infected eye between corneal surface and the lower eyelid. The composition would be administered four times per day for a period of two weeks. After five days of treatment, conjunctival erythema would begin to improve and the dendritiform lesions attenuate. By second week, these lesions have completely resolved, the cornea has re-epithelialized and would be clear and compact. The ocular examination would be otherwise unremarkable. The patient endorses complete symptomatic relief and returns to her normal functions.

(13) A 68-year-old female would present with visible, unilateral eye discomfort, pain and redness. She remembers a similar episode occurring a few years ago and states she has a history of ocular herpetic infections. Ophthalmoscopy would reveal 2+ erythematous conjunctiva without discharge. Corneal inspection would reveal an intact corneal epithelium; however, the corneal stroma demonstrates 2+ edema presenting in nummular fashion with underlying keratic precipitates. The patient receives a diagnosis of herpes simplex virus stromal and endothelial keratitis and a dosing regimen would be employed whereby an approximately ½-inch ribbon or gel drop of a composition comprising 0.50% PVP-I and 44% DMSO with 0.75% HEC would be administered directly to the surface of the infected eye between corneal surface and the lower eyelid. The composition would be administered four times per day for a period of two weeks. After five days of treatment, ocular erythema would begin to improve, and the stromal edema would begin to reverse. By second week, the endothelial changes have reversed and the keratic precipitates are no longer present. The ocular examination would be otherwise unremarkable. The patient endorses complete symptomatic relief and returns to her normal functions.

(14) A 26-year-old female would present with visible, bilateral eye discomfort, itching, redness and eyelid inflammation. She endorses a past medical history of HIV and would be immunocompromised. Ophthalmoscopy would reveal bilateral trace erythematous conjunctiva without chemosis or discharge. Eyelid inspection would reveal 1+ eyelid edema with erythema and multiple dome-shaped papules with central umbilication present on both lower eyelids. The patient receives a diagnosis of molluscum contagiosum and a dosing regimen would be employed whereby an approximately ½-inch ribbon or gel drop of a composition comprising 0.50% PVP-I and 44% DMSO with 1.50% HEC would be administered directly to the skin of the infected eyelid at the lower eyelid margin. The composition would be administered two times per day for a period of fourteen days. After five days of treatment, both the conjunctival and eyelid erythema would begin to improve. By the eighth day, the umbilicated eyelid papules have begun to involute and the end of the treatment cycle they are no longer present. The rest of the ocular exam would be unremarkable, and the patient endorses complete symptomatic relief.

(15) A 77-year-old male would present with a unilateral mass located near the limbus. Ophthalmoscopy would reveal injected conjunctiva with a leukoplakic growth encompassing both the conjunctiva and cornea that stains with rose bengal. Biopsy would reveal a corneal and conjunctival intraepithelial neoplasia consistent with carcinoma in situ. The patient receives a diagnosis of CIN and a dosing regimen would be employed whereby an approximately ½-inch ribbon or gel drop of a composition comprising 0.25% PVP-I and 44% DMSO with 1.5% HEC would be administered directly to the surface of the infected eye between corneal surface and the lower eyelid. The composition would be administered four times per day for a period of three months. After one week of treatment, the erythema would begin to improve. By the first month, the mass has significantly decreased in size. By the second month, there are only a few remaining areas of irregular conjunctiva. By the third month, the conjunctiva would be clear, and the ocular examination would be unremarkable. The patient endorses complete symptomatic relief and returns to her normal functions. Post treatment biopsy would be negative for neoplasia.

(16) A 66-year-old female would present with visible, bilateral eye discomfort, pain, redness and foreign body sensation. Ophthalmoscopy would reveal bilateral 1+ erythematous conjunctiva with punctate epithelial erosions and decreased Schirmer' s testing. Eyelid inspection would be within normal limits. The patient receives a diagnosis of aqueous deficiency dry eye and a dosing regimen would be employed whereby an approximately ½-inch ribbon or gel drop of a composition comprising 0.25% PVP-I and 44% DMSO with 0.25% HEC would be administered directly to the surface of the infected eye between corneal surface and the lower eyelid. The composition would be administered two times per day for a period of one month. After five days of treatment, the conjunctival erythema would begin to improve, as do the patient's symptoms. By the second week, the corneal punctate epithelial erosions are resolved. At one month, the Schirmer's testing has normalized and the patient experiences complete resolution of symptoms.

(17) A 72-year-old female would present with visible, bilateral eye discomfort, pain, redness and foreign body sensation. Ophthalmoscopy would reveal bilateral 1+ erythematous conjunctiva with punctate epithelial erosions and normal Schirmer's testing and decreased tear break up time. Eyelid inspection would be positive for mild meibomian gland capping with inspissation. The patient receives a diagnosis of evaporative dry eye and a dosing regimen would be employed whereby an approximately ½-inch ribbon or gel drop of a composition comprising 0.25% PVP-I and 44% DMSO with 0.50% HEC would be administered directly to the surface of the infected eye between corneal surface and the lower eyelid. The composition would be administered two times per day for a period of one month. After five days of treatment, the conjunctival erythema would begin to improve, as do the patient's symptoms. By the second week, the corneal punctate epithelial erosions are resolved. At one month, the tear break-up testing has normalized and the patient experiences complete resolution of symptoms.

(18) An 86-year-old male with wet macular degeneration would present to his ophthalmologist for intravitreal injection with an anti-VEGF agent. Ophthalmoscopy would reveal mild bilateral blepharitis and posterior segment would be positive for a choroidal neovascular complex. Eyelid inspection would be within normal limits. The patient receives a diagnosis of wet macular degeneration and in order to sterilize the ocular surface prior to the intravitreal injection a dosing regimen would be employed whereby an approximately ½-inch ribbon or gel drop of a composition comprising 0.50% PVP-I and 44% DMSO with 0.75% HEC would be administered directly to the surface of the eye between corneal surface and the lower eyelid prior to the procedure. This would be repeated four times while the eye would be in upgaze, downgaze, adduction and abduction. The procedure would be then performed without complications or infection.

(19) An 86-year-old male with wet macular degeneration would present to his ophthalmologist for removal of mass of the eyelid. Ophthalmoscopy would reveal mild bilateral blepharitis and a pedunculated neoplasia that encompasses part of the eyelid. The patient receives a diagnosis of a neoplastic ocular mass would be scheduled for surgical excision. In order to sterilize the eyelids, eyelashes and cheek prior to the procedure a dosing regimen would be employed whereby an approximately an ½-inch ribbon or gel drop of a composition comprising 0.50% PVP-I and 44% DMSO with 1.75% HEC would be administered directly to eyelids, eyelashes and cheek. This would be repeated two times while the eye would be in upgaze, downgaze, adduction and abduction. The procedure would be then performed without complications or infection.

(20) An 89-year-old female with wet macular degeneration would present to her ophthalmologist for cataract surgery. Previous ophthalmoscopy revealed mild bilateral blepharitis and 3+ nuclear sclerotic cataracts. Eyelid inspection was within normal limits. The patient received a diagnosis of mature, visually significant cataract and in order to sterilize the ocular surface prior to the cataract surgery a dosing regimen would be employed whereby an approximately ½-inch ribbon or gel drop of a composition comprising 0.50% PVP-I and 44% DMSO with 0.50% HEC would be administered directly to the surface of the eye between corneal surface and the lower eyelid prior to the procedure. This would be repeated four times while the eye would be in upgaze, downgaze, adduction and abduction. The procedure would be then performed without complications or infection.

While the foregoing written description enables a person ordinarily skilled in the art to reproduce and use what is considered presently to be the best mode thereof, those of ordinary skill will understand and appreciate the existence of variations, combinations, derivatives, analogs and equivalents of the specific embodiments, methods and examples provided above. The invention should therefore not be limited by the above described.

1. A stable gel composition comprising
0.15% to 1.5% povidone-iodine (PVP-I);
10% to 97% dimethyl sulfoxide (DMSO);
0.25% to less than 2.0% gelling agent; and
water or isotonic co-solvent;
wherein, the composition has a pH less than 4.5, and is free of additional anti-inflammatory drug.

2. The stable gel composition of claim 1, comprising 0.15% to 1.25% PVP-I.

3. The stable gel composition of claim 1, comprising 0.25% to 1.0% PVP-I.

4. The stable gel composition of claim 1, comprising 10% to 70% DMSO.

5. The stable gel composition of claim 1, comprising 30% to 49% DMSO.

6. The stable gel composition of claim 1, comprising about 0.25% gelling agent.

7. The stable gel composition of claim 1, comprising about 0.50% gelling agent.

8. The stable gel composition of claim 1, comprising about 1.0% gelling agent.

9. The stable gel composition of claim 1, comprising about 1.25% gelling agent.

10. The stable gel composition of claim 1, comprising about 1.50% gelling agent.

11. The stable gel composition of claim 1 comprising 1.75% gelling agent.

12. The stable gel composition of claim 1 wherein the gelling agent is a cellulosic polymer.

13. The stable gel composition of claim 1, comprising:
0.25% PVP-I;
44% DMSO;
greater than 0.25% and less than 2.0% HEC; and
a co-solvent; and
wherein said composition is steroid-free and NSAID-free.

14. The stable gel composition of claim 13, wherein the co-solvent is water or aqueous isotonic solution.

15. The stable gel composition of claim 1,
wherein, the stable gel composition is a topical ophthalmic preparation;
wherein each ingredient is ophthalmically acceptable; and
wherein said stable gel composition retains at least 85% of titratable iodine in povidone-iodine starting material for at least 72 hours.

16. The stable gel composition of claim 1 wherein the stable gel composition retains at least 85% of titratable iodine in povidone-iodine starting material for at least one month.

17. The stable gel composition of claim 1 wherein the stable gel composition retains at least 85% of titratable iodine in povidone-iodine starting material for up to 12 months.

18. A method of preventing an infectious condition of the eye or eyelid, said method comprising the step of:
applying an effective amount of a stable, topical ophthalmic gel composition of claim 1 to an eye or eyelid or periocular skin surface prior to surgery or invasive procedure of the eye including intraocular or intravitreal injection.

19. The method of claim 18 wherein said infectious condition is selected from the group consisting of blepharitis, conjunctivitis, corneal ulcer, bacterial keratitis, viral keratitis, post-operative endophthalmitis, and endophthalmitis after intravitreal or intracameral injection.

20. The method of claim 18, wherein the infectious condition is caused by or associated with one or more infectious agents selected from the group consisting of bacteria, demodex, fungus or yeast, and virus.

21. The method of claim 18 wherein the condition is blepharitis, blepharoconjunctivitis, conjunctivitis, keratitis or infectious corneal ulcer.

22. The method of claim 20, wherein the infectious agent is a virus.

23. The method of claim 20, wherein the infectious agent is demodex.

24. A method of treating an infectious condition of the eye or eyelid, said method comprising the step of:
applying an effective amount of a stable, topical ophthalmic gel composition of claim 1 to a site of the infection as needed to reduce or eliminate the infection.

25. The method of claim 24 wherein said infectious condition is selected from the group consisting of blepharitis, conjunctivitis, corneal ulcer, bacterial keratitis, viral keratitis, post-operative endophthalmitis, and endophthalmitis after intravitreal or intracameral injection.

26. The method of claim 24, wherein the infectious condition is caused by or associated with one or more infectious agents selected from the group consisting of bacteria, demodex, fungus or yeast, and virus.

27. The method of claim 24 wherein the condition is blepharitis, blepharoconjunctivitis, conjunctivitis, keratitis or infectious corneal ulcer.

28. The method of claim 26, wherein the infectious agent is a virus.

29. The method of claim 26, wherein the infectious agent is demodex.
